(19) United States
(12) Patent Application Publication
(10) Pub. No.: US 2005/0014722 A1
(43) Pub. Date: Jan. 20, 2005

(54) Process for preparing n-acylated lysophosphatidylcholine and pharmaceutical composition for treatment of metabolic bone disease comprising said compounds

(76) Inventors: Gil-Ja Jhon, Seoul (KR); So-Yeon Han, Seoul (KR); Zang-Hee Lee, Gwangiu-si (KR); Hong-Hee Kim, Gwangiu-si (KR); Eun-Hee Lee, Seoul (KR); Young-Ah Kim, Seoul (KR); Han-Bok Kwak, Gwangiu-si (KR)

Correspondence Address: MUSERLIAN, LUCAS AND MERCANTI, LLP, 475 PARK AVENUE SOUTH, 15TH FLOOR, NEW YORK, NY, 10016, US

(21) Appl. No.: 10/497,893

(22) PCT Filed: Dec. 13, 2002
(86) PCT No.: PCT/KR02/02357

(30) Foreign Application Priority Data
Dec. 13, 2001 (KR) 2001/79100

(51) Int. Cl. A61K 31/685

(52) U.S. Cl. 514/78

(57) ABSTRACT

Disclosed is a pharmaceutical composition for treating and preventing metabolic bone diseases, comprising a pharmaceutically effective amount of N-acylated lysophosphatidylcholine compound represented by Formula 1 (R is a saturated or unsaturated fatty acid of C14 to C20, and R′ is methoxycarbonyl or hydroxylmethyl group), and a pharmaceutically acceptable carrier. Also, the present invention discloses a method of preparing an N-acylated lysophosphatidylcholine compound represented by Formula 1, from serine. [Formula 1]

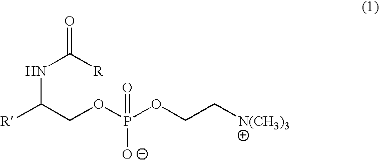

### TECHNICAL FIELD

The present invention relates to a pharmaceutical composition for treating and preventing metabolic bone diseases, comprising a pharmaceutically effective amount of a N-acylated lysophosphatidylcholine compound represented by Formula 1, below, and a pharmaceutically acceptable carrier.

### PRIOR ART

The skeleton consists of highly specialized bone cells including osteocytes, osteoclasts and osteoblasts, bone matrix including hydroxyapatite crystal, collagenous fibers and glycosaminoglycans, and spaces including bone marrow cavities, vascular canals, canaliculi and lacunae (Stavros C. M., Endocrine Reviews, 21(2), 115-137 (2000)). Bone functions to mechanically support the body, protect major organs, supply microenvironment required for hemopoiesis, and store calcium and several minerals.

Growth, development and maintenance of bone continue throughout life. Old bone is destroyed, and new bone is regenerated, replacing old bone. Such bone turnover occurs mainly at the basic multicellular unit comprising osteoclasts and osteoblasts. Bone remodeling serves to repair fine damage by growth and stress, and maintain function of bone. Destruction or resorption of old bone is accomplished by osteoclasts. In contrast, osteoblasts are responsible for formation of new bone.

Osteoclasts remove bone matrix such as hydroxyapaptite crystal or collagenous fibers, which constitute bone, by adhering to the bone surface and secreting hydrochloric acid and proteases. Osteoblasts synthesize and secrete bone matrix, and regulate the local concentration of calcium and phosphate to form skeleton (Stavros C. M., Endocrine Reviews, 21(2), 115-137 (2000)).

Metabolic bone diseases are caused by breakdown of the balance between osteoclasts and osteoblasts in the body. A representative example of such diseases is osteoporosis. Osteoporosis occurs due to reduction of total bone mass, resulting from both the excessive osteoclast activity and insufficient osteoblast activity. In osteoporosis, width of cortical bone is reduced, bone marrow cavity is enlarged, and thickness of trabecular bone is lowered, causing bone to be continuously porous. With progress of osteoporosis, physical strength of bone decreases, and thus lumbago and arthralgia are induced, and bone is easily fractured even by weak impact. In addition to osteoprosis, metabolic bone diseases include metastatic bone lesions caused by metastasis of breast and prostate carcinomas to bone, primary tumors of bone (e.g., multiple myeloma), rheumatoid or degenerative arthritis, periodontal disease accompanying destruction of alveolar bone by periodontal disease-causing bacteria, inflammatory periodontal disease with alveolar bone destruction generated after surgical application of dental implant, inflammatory bone resorption disease caused by implant implanted to fix bone by plastic surgery, and Paget's disease induced by various genetic factors.

Myeloma is a bone disease featured by fragile bone that is easily fractured, accompanying severe pain, and caused by osteoclast activity increased by carcinomas.

Breast and prostate carcinomas easily metastasize to bone, and stimulate osteoclast activity, resulting in destruction of bone. In case of rheumatoid or degenerative arthritis, tumor necrosis factor (TNF), interleukin-1 and interleukin-6, which are produced by the immune response, stimulate osteoclast activity present at the joint space, causing local destruction of bone at the joint. When inflammation is induced by infection with periodontal disease-causing bacteria, inflammatory cytokines including TNF, interleukin-1 and interleukin-6, produced by the immune response to the pathogenic bacterial infection, stimulate differentiation of osteoclasts, leading to destruction of alveolar bone supporting teeth.

With recently active molecular biological research related to therapy of metabolic bone diseases including osteoporosis, bone formation-stimulating factors and osteoclast-suppressing factors were developed. The bone formation-stimulating agents include fluoride, parathyroid hormone, TGF-β, bone morphogenetic protein, and insulin-like growth factor. Osteoclast-suppressing factors include estrogen, calcitonin, vitamin D and its analogues, and bisphosphonates (Jardine et al., Annual Reports in Medicinal Chemistry, 31, 211 (1996)).

Until now, several therapeutic agents for osteoporosis have been developed.

Among them, estrogen, which is most frequently used for treating osteoporosis, has disadvantages, as follows: it is still not demonstrated to be practically effective in treating osteoporosis, it should be administered throughout the patient's life, and its has side effects of increasing the incidence of breast cancer or cervical cancer when administered for a long period of time. Alendronate is also problematic in terms of being not clearly identified for its therapeutic efficacy for osteoporosis, being slowly absorbed by the gastrointestinal tract, and causing inflammation in the stomach, the intestine and mucosa of the esophagus. Calcium preparations are known to have mild side effects and good efficacy, but are a preventive agent rather than a therapeutic agent.

In addition, vitamin D such as calcitonin, which is used for preventing or treating osteoporosis, is not sufficiently studied for its preventive or therapeutic efficacy and side effects.

Therefore, there is a need for development of new therapeutic agents for bone-related diseases having few side effects and excellent therapeutic efficacy.

### DISCLOSURE OF THE INVENTION

In an aspect of the present invention, there is provided a pharmaceutical composition useful for treating and preventing metabolic bone diseases, comprising a pharmaceutically effective amount of a N-acylated lysophosphatidylcholine compound represented by Formula 1, below, and a pharmaceutically acceptable carrier.

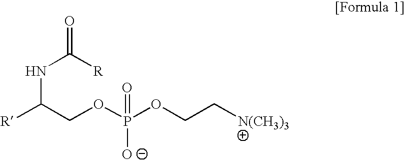

wherein, R is a saturated or unsaturated fatty acid having 14 to 20 carbon atoms, and R′ is methoxycarbonyl or hydroxylmethyl group.

In another aspect of the present invention, there is provided a method of preparing a N-acylated lysophosphatidylcholine compound represented by Formula 1 from an amino acid, serine.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing effect of CHJ-0014 as an example compound of the present invention on differentiation of osteoclasts in co-culture of bone marrow cells and osteoblasts;

FIG. 2 is a graph showing effect of CHJ-0014 as an illustrative compound of the present invention on differentiation of osteoclasts in culture of bone marrow cells;

FIG. 3 is a graph showing effect of CHJ-0014 as an illustrative compound of the present invention on differentiation of osteoclast precursor cells;

FIG. 4 is a graph showing cytotoxic activity of CHJ-0014 as an illustrative compound of the present invention to osteoblasts;

FIG. 5 is a graph showing cytotoxic activity of CHJ-0014 as an illustrative compound of the present invention to bone marrow cells;

FIG. 6 is a graph showing cytotoxic activity of CHJ-0014 as an illustrative compound of the present invention to peritoneal macrophages;

FIG. 7 is a graph showing cytotoxic activity of CHJ-0014 as an illustrative compound of the present invention to the human embryonic kidney cell line 293T;

FIG. 8 is a graph showing effect of CHJ-0014 as an illustrative compound of the present invention on the activity of the transcription factor NF-κB; and

FIG. 9 is a graph showing effect of CHJ-0013 and CHJ-0014 as illustrative compounds of the present invention on differentiation of osteoclasts in co-culture of bone marrow cells and osteoblasts.

### BEST MODES FOR CARRYING OUT THE INVENTION

Osteoclast progenitors are hematopoietic cells belonging to the monocyte/macrophage lineage originating in bone marrow. Osteoclast progenitors differentiate and develop by growth factors and cytokines produced in bone marrow (Roodman G. D., Endocr. Rev., 17, 308-332 (1996)). Osteoclasts serve to destroy or resorb bone.

Recently, osteoclast differentiation factor (ODF), required for differentiation of osteoclasts, was cloned. ODF is a new member of the tumor necrosis factor (TNF) ligand family, and associated to the plasma membrane. Soluble ODF, which is prepared by a genetic engineering method, was reported to stimulate formation of osteoclasts in the presence of macrophage colony stimulating factor (M-CSF) without any help of osteoblasts or stromal cells. ODF is also called TRANCE, OPGL or RANKL. ODF displays its function through binding to RANK, which is a TNF receptor family member present in osteoclast precursors and mature osteoclasts. In mice, expression of ODF is limited to bone, spleen, thymus and lung. ODF was reported to be increasingly expressed under condition of high bone resorption in cultured osteoblasts (Suda T. et. al., Endocr. Rev., 20, 345-357 (1999); and Yasuda et al., Proc. Natl. Acad. Sci. USA, 95(7), 3597-3604 (1998)).

Osteoclastogenesis-inhibitory factor (OCIF), also called osteoprotogerin (OPG), inhibits production of osteoclasts and activity of mature osteoclasts. OPG is a secreted TNF receptor, and binds with high affinity to ODF associated with cells. Bone regeneration is regulated by ODF and OPG. Mature osteoclasts are multinucleated cells about 50-100 μm in diameter and have a morphological character of wrinkled surface, and play a role to resorb calcified bone matrix (Boskey A. L., J. Cell. Biochem. Suppl., 30-31, 83-91 (1998)). Mature osteoclasts adhere to the surface of bone matrix, secrete proteases and an acidic material into the sealing zone between the plasma membrane of osteoclasts and bone matrix, and eventually destroy bone by acidification and proteolytic digestion.

The N-acylated lysophosphatidylcholine compound of Formula 1, above, used as an active ingredient in treating and preventing metabolic bone diseases according to the present invention, comprises compounds represented by Formulas 1a to 1d, below.

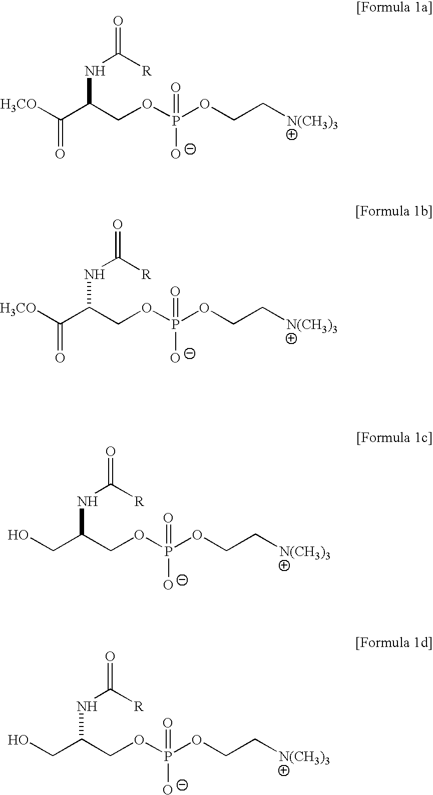

wherein, R is a saturated or unsaturated fatty acid having 14 to 20 carbon atoms.

The compound of Formula 1 according to the present invention may be prepared by a process comprising esterification, amide bond formation, phosphorylcholine preparation and reduction. In detail, a method of preparing the compounds of Formulas 1A to 1D according to the present invention comprises the steps of:
(a) reacting serine with methanol and hydrochloric acid to produce serine methylester hydrochloride (esterification);
(b) reacting the serine methylester hydrochloride with N-methylmorpholine, a saturated or unsaturated fatty acid having 14 to 20 carbon atoms, 1-hydroxybenzotriazole and 1,3-dicyclohexylcarbodiimide to produce N-acyl-serine methylester (amide bond formation);
(c) reacting the N-acyl-serine methylester with N-diisopropylethyl amine and ethylene chlorophosphite, and then trimethylamine to produce N-acyl-O-phosphocholine-serine methylester (pliosphocholine preparation); and
(d) reacting the N-acyl-O-phosphocholine-serine methylester with lithiumaluminumhydride to produce N-acyl-O-phosphocholine-serine methylhydroxy (reduction).

A compound having a hydroxymethyl R′ group according to the present invention may be prepared using an amino acid, serine, as a starting material according to a method as disclosed in Korean Pat. Publication No. 2000-59468, comprising esterification, amide bond formation, phosphocholine preparation and reduction, as shown in the following Reaction Formula:

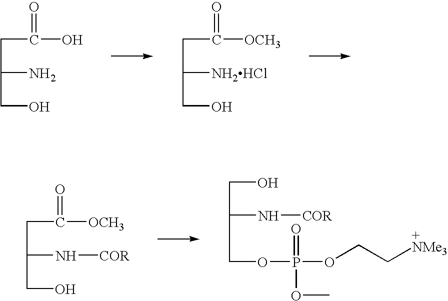

wherein, R is a saturated or unsaturated fatty acid having 14 to 20 carbon atoms.

In the compound of Formula 1, used as an active ingredient in the pharmaceutical composition according to the present invention, the fatty acid designated ‘R’ includes unsaturated fatty acids having 14 to 20 carbon atoms, and non-limiting examples of the unsaturated fatty acids may include fatty acids of C16:1, C18:1, C18:2 and C20:4.

In the compound of Formula 1, used as an active ingredient in the pharmaceutical composition according to the present invention, the fatty acid designated ‘R’ includes saturated fatty acids having 14 to 20 carbon atoms, and a representative example of the saturated fatty acids is stearic acid having 17 carbon atoms. In the case that R is stearic acid, the N-acylated lysophosphatidylcholine compound of Formula 1 comprises compounds represented by the following Formulas:

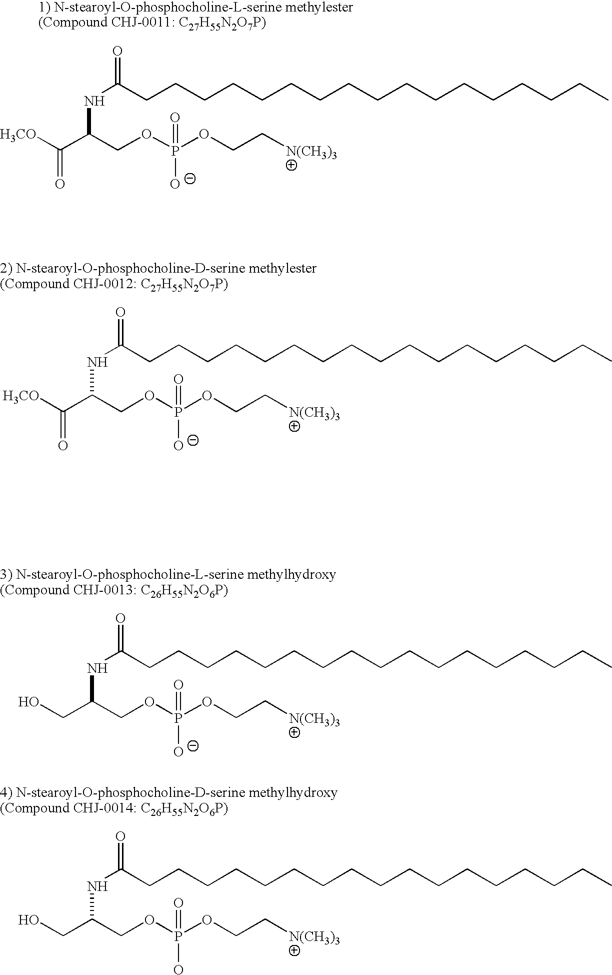

The illustrative compounds CHJ-0013 and CHJ-0014 may be prepared, as described above, using serine as a starting material according to a method as disclosed in Korean Pat. Publication No. 2000-59468, comprising esterification, amide bond formation, phosphocholine preparation and reduction. In addition, the illustrative compounds of the Formula 1, CHJ-0011, CHJ-0012, CHJ-0013 and CHJ-0014 may be prepared according to the method comprising esterification, amide bond formation, phosphocholine preparation and reduction according to the present invention.

The compound of the Formula 1, prepared according to the method of the present invention, includes its D-form and L-form stereoisomers, and may be stereo-selectively synthesized according to the method of the present invention. For example, in case of using L-serine as a starting material, a L-form final compound is specifically produced, which is exemplified by the CHJ-0011 and CHJ-0013 compounds. In case of using D-serine as a starting material, a D-form final compound is specifically generated, which is exemplified by the CHJ-0012 and CHJ-0014 compounds.

Therefore, in an additional aspect, the present invention provides a method of preparing a compound represented by the Formula 1, which is in a L-stereoisomeric form, comprising the steps of: (a) reacting L-serine with methanol and hydrochloric acid to produce L-serine methylester hydrochloride; (b) reacting the L-serine methylester hydrochloride with N-methylmorpholine, a saturated or unsaturated fatty acid having 14 to 20 carbon atoms, 1-hydroxybenzotriazole and 1,3-dicyclohexylcarbodiimide to produce N-acyl-L-serine methylester; (c) reacting the N-acyl-L-serine methylester with N-diisopropylethyl amine and ethylene chlorophosphite, and then trimethylamine to produce N-acyl-O-phosphocholine-L-serine methylester; and (d) reacting the N-acyl-O-phosphocholine-L-serine methylester with lithiumaluminumhydride to produce N-acyl-O-phosphocholine-L-serine methylhydroxy.

In a further additional aspect, the present invention provides a method of preparing a compound represented by the Formula 1, which is in a D-stereoisomeric form, comprising the steps of: (a) reacting D-serine with methanol and hydrochloric acid to produce D-serine methylester hydrochloride; (b) reacting the D-serine methylester hydrochloride with N-methylmorpholine, a saturated or unsaturated fatty acid having 14 to 20 carbon atoms, 1-hydroxybenzotriazole and 1,3-dicyclohexylcarbodiimide to produce N-acyl-D-serine methylester; (c) reacting the N-acyl-D-serine methylester with N-diisopropylethyl amine and ethylene chlorophosphite, and then trimethylamine to produce N-acyl-O-phosphocholine-D-serine methylester; and (d) reacting the N-acyl-O-phosphocholine-D-serine methylester with lithiumaluminumhydride to produce N-acyl-O-phosphocholine-D-serine methylhydroxy.

In an aspect of the present invention, there is provided a method of preparing a compound represented by the Formula 1, which is in a L-stereoisomeric form, comprising the steps of: (a) reacting L-serine with methanol and hydrochloric acid to produce L-serine methylester hydrochloride; (b) reacting the L-serine methylester hydrochloride with N-methylmorpholine, stearic acid, 1-hydroxybenzotriazole and 1,3-dicyclohexylcarbodiimide to produce N-stearoyl-L-serine methylester; (c) reacting the N-stearoyl-L-serine methylester with N-diisopropylethyl amine and ethylene chlorophosphite, and then trimetbylamine to produce N-stearoyl-O-phosphocholine-L-serine methylester; and (d) reacting the N-stearoyl-O-phosphocholine-L-serine methylester with lithiumaluminumhydride to produce N-stearoyl-O-phosphocholine-L-serine methylhydroxy.

In another aspect of the present invention, there is provided a method of preparing a compound represented by the Formula 1, which is in a D-stereoisomeric form, comprising the steps of: (a) reacting D-serine with methanol and hydrochloric acid to produce D-serine methylester hydrochloride; (b) reacting the D-serine methylester hydrochloride with N-methylmorpholine, stearic acid, 1-hydroxybenzotriazole and 1,3-dicyclohexylcarbodiimide to produce N-stearoyl-D-serine methylester; (c) reacting the N-stearoyl-D-serine methylester with N-diisopropylethyl amine and ethylene chlorophosphite, and then trimethylamine to produce N-stearoyl-O-phosphocholine-D-serine methylester; and (d) reacting the N-stearoyl-O-phosphocholine-D-serine methylester with lithiumaluminumhydride to produce N-stearoyl-O-phosphocholine-D-serine methylhydroxy.

In practical application of the method according to the present invention, detailed reaction conditions may follow the conventional conditions common in the art.

Amino acid methylester hydrochloride may be produced by a method common in the art. That is, an amino acid is reacted with methanol saturated with hydrochloric acid gas to weaken nucleophilic property of an amino group of the amino acid, and a carboxyl group of the amino acid is then selectively methyl-esterificated. Therefore, after reacting L-serine with methanol saturated with hydrochloric acid gas at room temperature for 2 hrs, the reaction product may be easily purified by recrystallization using ether/methanol.

Amide bond formation may be achieved by activating the carboxyl group with a proper peptide bond-forming reagent. In particular, because of having a primary amino group, L-serine methylester hydroxychloride is much more reactive than compounds having secondary amino groups. Therefore, even when using the relatively cheap 1,3-dicyclohexylcarbodiimide as a peptide bond-forming agent, the reaction product can be obtained at high yield. Herein, when 1,3-dicyclohexylcarbodiimide is used in combination with the racemization-inhibiting agent 1-hydroxybenzotriazole, the reaction product is stereo-selectively synthesized. This method is described in the reference incorporated in the present invention: Tetrahedron Lett. 1996, 37, 2083-2084.

The phosphocholination reaction may be carried out by reaction with ethylene chlorophosphite and sequentially aqueous phase trimethylamine. Typically, phosphorylation can be achieved by using ethylene chlorophosphite, 2-chloro-2-oxo-1,2,3-dioxaphosphorane or 2-bromoethyldichlorophosphate, but the above method gives the most effective result. N-stearoyl-L-serine methylester is dissolved in tetrahydrofuran, and reacted with N-diisopropylethyl amine and ethylene chlorophosphite. Then, bromine and water are added to the reaction mixture, and the formed compound is recrystallized with dichloromethane/acetone. The recrystallized compound is dissolved in chloroform/isopropanol/acetonitrile, and aqueous phase trimethylamine is added to the resulting solution to form a phosphocholine region.

This method is described in the reference incorporated in the present invention: J. Org. Chem. 1998, 63, 2560-2563. The reduction step at which the methylester group of N-stearoyl-O-phosphocholine-L-serine methylester is converted to a hydroxyl group may be accomplished by using a typical reducing agent, lithiumaluminumhydride. This method described in the reference incorporated in the present invention: Tetrahedron Lett. 2001, 42, 5645-5649.

The active ingredient used in the pharmaceutical composition of the present invention comprises “a pharmaceutically acceptable salt” of the compound of Formula 1. The salt is prepared by reaction with a stoichiometric amount of a suitable base or acid in water or an organic solvent, or in a mixture of the two. Typically, the pharmaceutically acceptable salt useful in the present invention includes inorganic base salts, organic base salts, inorganic acid salts, organic acid salts, and basic or acidic amino acid salts. Examples of the inorganic base salts include alkali metal salts such as sodium salts or potassium salts, alkali earth metal such as calcium salts or magnesium salts, aluminum salts, and ammonium salts. Examples of the organic base salts include salts of trimethylamine, triethylamine, pyridine, picoline, 2,6-lutidine, ethanolamine, diethanolamine, triethanolamine, cyclohexylamine, dicyclohexylamine and N,N′-dibenzylethylenediamine. Examples of the organic acid salts include salts of formic acid, acetic acid, trifluoroacetic acid, phthalic acid, fumaric acid, oxalic acid, tartaric acid, maleic acid, citric acid, succinic acid, malic acid, methanesulfonic acid, benzensulfonic acid and ρ-toluenesulfonic acid. Examples of the basic amino acid salts include salts of arginine, lysine and ornithine. Examples of the acidic amino acid salts include salts of aspartic acid and glutamic acid. The salts of the present invention may be prepared by conventional methods such as ion exchange. Suitable salts are summarized in the reference incorporated in the present invention: Remington's Pharmaceutical Sciences, 17th ed., Mack Publishing Company, Easton, Pa., 1985, p1418.

The compound of Formula 1 according to the present invention inhibits differentiation of osteoclasts both in co-culture of bone marrow cells and osteoblasts and in culture of bone marrow cells. The compound of Formula 1 inhibits differentiation of osteoclasts during differentiation of osteoclat precursors isolated from bone in a dose-dependent manner. In addition, the compound of Formula 1 inhibits activation of the transcription factor NF-κB by osteoclast differentiation factor (ODF).

Moreover, the compound of Formula 1 does not have cytotoxic activity to osteoblasts, bone marrow cells, peritoneal macrophages and kidney cells. Therefore, the compound of the present invention and its pharmaceutically acceptable salts may be used separately or as a mixture of over two components for preventing and treating metabolic bone diseases. The term “metabolic bone disease” refers to a disease accompanying a physiopathological state caused by excessive destruction and resorption of bone. Examples of the metabolic bone diseases include osteoporosis, metastatic bone lesions caused by metastasis of breast or prostate carcinomas to bone, primary bone tumors (e.g., multiple myeloma), rheumatoid or degenerative arthritis, periodontal disease accompanying destruction of alveolar bone by periodontal disease-causing bacteria, inflammatory periodontal disease with alveolar bone destruction generated after surgical application of dental implant, inflammatory bone resorption disease caused by implant implanted to fix bone by plastic surgery, and Paget's disease induced by various genetic factors.

Such bone diseases are induced mainly by increased bone resorption. For this reason, development of therapeutic agents for bone diseases focuses on reducing bone resorption (science 289, 2000(9), 1508-1514). Osteoclasts are responsible for bone resorption (Enclocr. Rev. 13, 1992, 66-80; and Bone, 17, 1995, 87s-91s). Therefore, with respect to established therapy of bone diseases, it is a major issue to develop agents having effects of inhibiting osteoclast formation and activity. Among drugs used as osteoporosis therapeutic agents, estrogen suppresses osteoclast formation (J. Biol. Chem. 276(23), 2001, 8836-8840; and Endocrinology 139,1998,3022-3025), and bisphosphonates and calcitonin inhibit osteoclast activity (science 289, 2000(9), 1508-1514; J. Biol. Chem. 274, 1999, 34967), resulting in reduced bone resorption.

In this respect, the N-acylated lysophosphatidylcholine compound represented by the Formula 1, which was found to have effects of strongly inhibiting osteoclast formation both in co-culture of bone marrow cells and osteoblasts and in culture of bone marrow cells, may be useful as a therapeutic agent for bone diseases. The preferred compound has a hydroxymethyl R′ group, and the most preferred compound, which has a stearic acid R group, is N-stearoyl-O-phosphocholine-L-serine methylhydroxy, and N-stearoyl-O-phosphocholine-D-serine methylhydroxy.

The compound of Formula 1 and its pharmaceutically acceptable salts may be used alone, or as a formulated form in combination with a pharmaceutically acceptable carrier, for preventing and treating metabolic bone diseases. The term “pharmaceutically acceptable carrier” means a pharmaceutically acceptable substance, composition or vehicle serving to deliver an active component from one organ or a portion of the body to another organ or a different region of the body, where the pharmaceutically acceptable substance is exemplified by liquid or solid fillers, diluents, excipients and solvents.

The pharmaceutical composition of the present invention may be administered orally, topically, or parenterally or by injection, and comprises the compound of Formula 1 as an effective ingredient at an amount of about 0.5-90 wt %, where the amount is therapeutically effective for metabolic bone diseases. Oral preparations according to the present invention may be administered in a pharmaceutical formulation, for example, pills, tablets, lacquered tablets, coated tablets, powder, granules, troches, wafers, elixirs, hard and soft gelatin capsules, solutions, syrups, emulsions, suspensions and spray mixtures. Examples of parenteral preparations may include injection preparations, microcapsules and transdermally administered preparations. A therapeutic agent for periodontal disease may be used as a delayed release delivery system or in a delayed release formulation in which dental implant is coated with a delayed release delivery substance.

The pharmaceutical composition may be formulated into a suitable pharmaceutical form by the known method employing an inert inorganic or organic excipient. For example, to formulate into pills, tablets, coated tablets and hard gelatin capsules, lactose or corn starch or derivatives thereof, talc, and stearic acid or its salts may be used. Excipients useful for preparation of soft gelatin capsules and suppositories are exemplified by fats, waxes, semi-solid and liquid polyols, and natural or hardened oils. Excipients suitable for preparation of solutions and syrups are exemplified by water, sucrose, invert sugars, glucose, and polyols. Excipients suitable for preparation of injection preparations are exemplified by water, alcohol, glycerol, polyols and vegetable oils. The injection preparations may be used in combination with preserving agents, analgesics, solubilizers and stabilizers.

Pharmaceutical preparations for local administration may be prepared in combination with bases, excipients, lubricants, preserving agents, and the like.

Excipients suitable for preparation of microcapsules or implants include copolymers, glycolic acid and lactic acid.

In addition to an active compound and excipients, the pharmaceutical preparation of the present invention may further comprise additives, which are exemplified by fillers, thickening agents, disintegrators, binders, lubricants, humectants, stabilizers, emulsifiers, antiseptics, sweetening agents, coloring agents, perfumes or aromatic agents, concentrating agents, diluents and buffering agent, other solvents or solubilizers, substances to obtain depot effect, and salts to change osmotic pressure, and coating agents or anti-oxidant agents.

In addition, the pharmaceutical preparation of the present invention may comprise two or more derivatives of the compound represented by Formula 1 or their pharmaceutically acceptable salts, and one or more of other therapeutically active substances. Examples of the other therapeutically active substances may include blood circulation-promoting agent (e.g., dihydroergocristine, nicergoline, buphenine, nicotinic acid and its ester, pyridilcarbinole, bencyclane, cinnarizine, naftidrofuryl, raubasine, vincamine), positive inotropic agents (e.g., digoxin, acethyldigoxine, methyldigoxine and lanato-glycoside), coronary vasodilators (e.g., carbocromen, dipyridamole, nifedipin, perhexiline), antianginals (e.g., isosorbid dinitrate, isosorbid mononitrate, glycerol nitrate, molsidomine, berapamil), and beta-blockers (e.g., propanolol, oxprenolol, atenolol, metoprolol, penbutolol). In addition, the pharmaceutical preparation of the present invention may comprise other nootropics (e.g., pyracetam), or the central nervous system-acting substances (e.g., pirlindole, sulpiride).

Administration dosage of the compound of Formula 1 according to the present invention may be suitably determined according to absorption rate of an active ingredient in the body, inactivation and excretion rates of the active ingredient, patient's age, sex and physical condition, and advanced state of a disease. Typically, daily dosage to obtain therapeutic efficacy for metabolic bone diseases may be, in case of oral administration, about 0.1-1 mg/kg body weight, and preferably, 0.3-0.5 mg/kg. Daily dosage for intravenous administration is typically about 0.01-0.3 mg/kg body weight, and preferably, 0.05-0.1 mg/kg. In particular, in case of being administered at a relatively large amount, daily dosage is commonly separately administered over several times per day, for example, 2, 3 or 4 times per day. The daily dosage may be increased or reduced according to individual cases.

The present invention will be explained in more detail with reference to the following an example in conjunction with the accompanying drawings. However, the following example is provided only to illustrate the present invention, and the present invention is not limited to the example.

### EXAMPLE 1

### Synthesis of N-stearoyl-O-phosphocholine-L-serine methylester (CHJ-0011)

(i) Synthesis of L-serine methyl ester hydrochloride

47.7 mmol of L-serine was dissolved in 476 ml of methanol, saturated with hydrochloric acid gas, and incubated at room temperature for 2 hrs. After evaporating the solvent, the reaction product was recrystallized from ether/methanol, thereby generating L-serine methylester hydrochloride (yield: 98%, melting point: 161-162° C., [α]²⁵D=+3.4(c 0.2, MeOH)). The structure of the final product was identified by FTIR, ¹H-NMR and ¹³C-NMR.

FTIR (KBr, cm⁻¹): 3349 O—H peak, 2943 sp³ C—H peak, 1749 ester carbonyl peak; and

¹H NMR (CD₃OD): δ 4.07~4.10(1H, t, J=3.9 Hz), 3.88-3.93(2H, m), 3.79 (3H, s) methoxy carbon proton (s: singlet, d: doublet, t: triplet, m: multiplet)

¹³C NMR (CD₃OD): δ52.69, 55.10, 59.67, 168.37 carbonyl peak.

(ii) Synthesis of N-stearoyl-L-serine methyl ester

The compound (1 eq) prepared in the above (i) was dissolved in 257 ml of dichloromethane, and cooled to 0° C. N-methylmorpholine (2.1 eq), stearic acid (1.1 eq) and 1-hydroxybenzotriazole (1.1 eq), and 1,3-dicyclohexyl carbodiimide (1.1 eq) were sequentially added to the cooled solution, and the mixture was incubated on ice for 1 hr, and then at room temperature for 3 hrs. Thereafter, the by-product dicyclourethane was filtered under pressure, and the remaining solution was concentrated. The resulting solution was subjected to column chromatography (dichloromethane: acetone=9:1→7:1), thereby purifying the reaction product N-stearoyl-L-serine methyl ester (yield: 90%, melting point: 81-82° C., [α]²⁵D=+15.2 (c 0.2, CHCl₃)). The structure of the final product was identified by FTIR, ¹H-NMR and ¹³C-NMR.

FTIR (KBr, cm⁻¹): 3310 O—H peak, 2919 sp³ C—H peak, 1720 ester carbonyl peak, 1650 amide carbonyl peak;

¹H NMR (CDCl₃): δ 0.83~0.88(3H, m) stearic acid terminal carbon proton, 1.23 (28H, s) hydrocarbon proton, 1.60~1.63(2H, m) carbonyl-β-carbon proton, 2.21~2.28 (2H, t, J=7.6 Hz), 2.52(1H, m) hydroxyl peak, 3.78(3H, s) methoxy carbon proton, 3.93~3.94 (2H, d, J=3.4 Hz), 4.64~4.70(1H, m), 6.36~6.39(1H, d, J=6.5 Hz) amide nitrogen proton; and

¹³C NMR (CDCl₃): δ 14.1 stearic acid terminal carbon, 22.7 hydrocarbon hydrocarbon proton, 25.5 carbonyl-β-carbon, 29.2, 29.3, 29.5, 29.7, 31.9 hydrocarbon, 36.5, 52.8 methoxy carbon, 54.6, 63.7, 171.0 carbonyl peak, 173.8 carbonyl peak.

(iii) Synthesis of N-stearoyl-O-phosphocholine-L-serine methyl ester (CHJ-0011)

The compound (1 eq) prepared in the above (ii) was dissolved in 260 ml of tetrahydrofuran, and cooled to −15° C. N-diisopropylethyl amine (4 eq) and ethylene chlorophosphite (3 eq) were added to the cooled solution, followed by incubation for 1 hr. After adding bromine (3 eq) to the mixture and incubated for 15 min, 86.6 ml of water was added to the reaction mixture, followed by incubation for 1 hr. The separated organic layer was evaporated, and the remaining solution was recrystallized from dichloromethane and acetone. The recrystallized product was again dissolved in 87.5 ml of chloroform/isopropanol/acetonitrile (3:5:5, v/v/v) at 0° C., supplemented with 40% aqueous phase trimethylamine, and incubated for 11 hrs. Thereafter, the reaction mixture was subjected to column chromatography (dichloromethane: methanol: water=3:1:0→2:1:0.1), thereby yielding N-stearoyl-O-phosphocholine-L-serine methyl ester (yield: 12%, [α]²⁵D=−8.4 (c 1.9, MeOH)). The structure of the purified product was identified by ¹H-NMR and ¹³C-NMR.

¹H NMR (CDCl₃): δ 0.90~0.93(3H, m) stearic acid terminal carbon proton, 1.31 (28H, s) hydrocarbon proton, 1.63~1.65(2H, m) carbonyl-β-carbon proton, 2.27~2.33 (2H, t, J=7.2 Hz), 3.25(9H, s) trimethylamine carbon proton, 3.65~3.67(2H, m), 3.77(3H, s) methoxy peak, 4.15~4.19(11H, m), 4.21~4.28(3H, m), 4.68(1H, m); and

¹³C NMR (CDCl₃): δ 13.5 stearic acid terminal carbon, 22.8 hydrocarbon proton, 25.9 carbonyl-β-carbon, 29.3, 29.5, 29.8, 32.1, 35.7, 51.9 methoxy carbon, 53.7, 59.5, 65.1, 66.4, 170.6 carbonyl peak, 175.4 carbonyl peak.

### EXAMPLE 2

### Synthesis of N-stearoyl-O-phosphocholine-L-serine methylhydroxy (CHJ-0013)

(i) Synthesis of L-serine methyl ester hydrochloride

47.7 mmol of L-serine was dissolved in 476 ml of methanol, saturated with hydrochloric acid gas, and incubated at room temperature for 2 hrs. After evaporating the solvent, the reaction product was recrystallized from ether/methanol, thereby generating L-serine methylester hydrochloride (yield: 98%, melting point: 161-162° C., [α]²⁵D=+3.4(c 0.2, MeOH)). The structure of the final product was identified by FTIR, ¹H-NMR and ¹³C-NMR.

FTIR (KBr, cm⁻¹): 3349 O—H peak, 2943 sp³ C—H peak, 1749 ester carbonyl peak; and

¹H NMR (CD₃OD): δ 4.07~4.10(1H, t, J=3.9 Hz), 3.88-3.93(2H, m), 3.79 (3H, s) methoxy carbon proton (s: singlet, d: doublet, t: triplet, m: multiplet)

¹³C NMR (CD₃OD): δ52.69, 55.10, 59.67, 168.37 carbonyl peak.

(ii) Synthesis of N-stearoyl-L-serine methyl ester

The compound (1 eq) prepared in the above (i) was dissolved in 257 ml of dichloromethane, and cooled to 0° C. N-methylmorpholine (2.1 eq), stearic acid (1.1 eq) and 1-hydroxybenzotriazole (1.1 eq), and 1,3-dicyclohexyl carbodiimide (1.1 eq) were sequentially added to the cooled solution, and the mixture was incubated for 1 hr, and then at room temperature for 3 hrs. Thereafter, the by-product dicyclourethane was filtered under pressure, and the remaining solution was concentrated. The resulting solution was subjected to column chromatography (dichloromethane: acetone=9:1→7:1), thereby purifying the reaction product N-stearoyl-L-serine methyl ester (yield: 90%, melting point: 81-82° C., [α]²⁵D=+15.2 (c 0.2, CHCl₃)). The structure of the final product was identified by FTIR, ¹H-NMR and ¹³C-NMR.

FTIR (KBr, cm⁻¹): 3310 O—H peak, 2919 Sp³ C—H peak, 1720 ester carbonyl peak, 1650 amide carbonyl peak;

¹H NMR (CDCl₃): δ 0.83~0.88(3H, m) stearic acid terminal carbon proton, 1.23 (28H, s) hydrocarbon proton, 1.60~1.63(2H, m) carbonyl-β-carbon proton, 2.21~2.28 (2H, t, J=7.6 Hz), 2.52(1H, m) hydroxyl peak, 3.78(3H, s) methoxy carbon proton, 3.93~3.94 (2H, d, J=3.4 Hz), 4.64~4.70(1H, m), 6.36~6.39(1H, d, J=6.5 Hz) amide nitrogen proton; and

¹³C NMR (CDCl₃): δ 14.1 stearic acid terminal carbon, 22.7 hydrocarbon hydrocarbon proton, 25.5 carbonyl-β-carbon, 29.2, 29.3, 29.5, 29.7, 31.9 hydrocarbon, 36.5, 52.8 methoxy carbon, 54.6, 63.7, 171.0 carbonyl peak, 173.8 carbonyl peak.

(iii) Synthesis of N-stearoyl-O-phosphocholine-L-serine methyl ester

The compound (1 eq) prepared in the above (ii) was dissolved in 260 ml of tetrahydrofuran, and cooled to −15° C. N-diisopropylethyl amine (4 eq) and ethylene chlorophosphate (3 eq) were added to the cooled solution, followed by incubation for 1 hr. After adding bromine (3 eq) to the mixture and incubated for 15 min, 86.6 ml of water was added to the reaction mixture, followed by incubation for 1 hr. The separated organic layer was evaporated, and the remaining solution was recrystallized from dichloromethane and acetone. The recrystallized product was again dissolved in 87.5 ml of chloroform/isopropanol/acetonitrile (3:5:5, v/v/v) at 0° C., supplemented with 40% aqueous phase trimethylamine, and incubated for 11 hrs. Thereafter, the reaction mixture was subjected to column chromatography (dichloromethane: methanol: water=3:1:0→2:1:0.1), thereby yielding N-stearoyl-O-phosphocholine-L-serine methyl ester (yield: 12%, [α]²⁵D=−8.4 (c 1.9, MeOH)). The structure of the purified product was identified by ¹H-NMR and ¹³C-NMR.

¹H NMR (CDCl₃): δ 0.90~0.93(3H, m) stearic acid terminal carbon proton, 1.31 (28H, s) hydrocarbon proton, 1.63~1.65(2H, m) carbonyl-β-carbon proton, 2.27~2.33 (2H, t, J=7.2 Hz), 3.25(9H, s) trimethylamine carbon proton, 3.65~3.67(2H, m), 3.77(3H, s) methoxy peak, 4.15~4.19(1H, m), 4.21~4.28(3H, m), 4.68(1H, m); and

¹³C NMR (CDCl₃): δ 13.5 stearic acid terminal carbon, 22.8 hydrocarbon proton, 25.9 carbonyl-β-carbon, 29.3, 29.5, 29.8, 32.1, 35.7, 51.9 methoxy carbon, 53.7, 59.5, 65.1, 66.4, 170.6 carbonyl peak, 175.4 carbonyl peak.

(iv) Synthesis of N-stearoyl-O-phosphocholine-Lserine methylhydroxy (CHJ-0013)

The compound (1 eq) prepared in the above (iii) was dissolved in 12 ml of tetrahydrofuran, and cooled to 0° C. Lithiumaluminumhydride (3 eq) were added to the cooled solution, followed by incubation for 4 hr. The reaction mixture was filtered under pressure, and the remaining solution was concentrated and freeze-dried.

Thereafter, the resulting solution was subjected to column chromatography (dichloromethane: methanol: water=3:1:0→2:1:0.1→1:1:0.3), thereby yielding N-stearoyl-O-phosphocholine-L-serine methylhydroxy (CHJ-0013) (yield: 54%, [α]²⁵D=+5.3 (c 1.9, CH₂Cl₂/MeOH)). The structure of the purified product was identified by FTIR, ¹H-NMR and ¹³C-NMR.

FTIR (KBr, cm⁻¹): 3266 O—H peak, 2920 sp³ C—H peak, 1654 amide carbonyl peak, 1236 phosphate ester peak;

¹H NMR (CD₃OD): δ 0.78~0.82(3H, m) stearic acid terminal carbon proton, 1.19 (28H, s) hydrocarbon proton, 1.51(2H, m) carbonyl-β-carbon proton, 2.09~2.15(2H, t, J=7.5 Hz), 3.13(9H, s) trimethylamine carbon proton, 3.50~3.56(4H, m),3.81~3.96(3H, m), 4.18~4.19(2H, m);

¹³C NMR (CD₃OD): δ 13.5 stearic acid terminal carbon, 22.8 hydrocarbon carbon, 26.1 carbonyl-β-carbon, 29.4, 29.5, 29.7, 29.8, 32.1 hydrocarbon carbon, 36.2, 51.6, 51.8, 53.7 trimethylamine carbon, 59.4, 59.5, 60.3, 64.0, 64.1, 66.4 phosphatidylcholine methyline carbon, 175.3 carbonyl peak; and

FABHRMS (m/z) [M+H]+calcd for C₂₆H₅₆N₂O₆P: 523.3876, found 523.3861.

### EXAMPLE 3

### Synthesis of N-stearoyl-O-phosphocholine-D-serine methylester (CHJ-0012)

(i) Synthesis of D-serine methyl ester hydrochloride

D-serine methyl ester hydrochloride was synthesized according to the same method as in Example 1 for synthesis of L-serine methyl ester hydrochloride, except for use of D-serine instead of L-serine (yield: 99%, melting point: 163-164° C., [α]²⁵D=−4.3 (c 1.8, EtOH)). When analyzing the structure of the synthesized compound, the FTIR, ¹H-NMR and ¹³C-NMR results were identical to those of L-serine methyl ester hydrochloride.

(ii) Synthesis of N-stearoyl-D-serine methyl ester

N-stearoyl-D-serine methyl ester was synthesized according to the same method as in Example 1 (ii) for synthesis of N-stearoyl-L-serine methyl ester, except for use of D-serine-methyl ester hydrochloride (yield: 88%, melting point: 82-83° C., [α]²⁵D=−15.7 (c 2.0, CHCl₃)). When analyzing the structure of the synthesized compound, the FTIR, ¹H-NMR and ¹³C-NMR results were identical to those of N-stearoyl-L-serine methyl ester.

(iii) Synthesis of N-stearoyl-O-phosphocholine-D-serine methyl ester (CHJ-0012)

N-stearoyl-O-phosphocholine-D-serine methyl ester was synthesized according to the same method as in Example 1 (iii) for synthesis of N-stearoyl-O-phosphocholine-L-serine methyl ester, except for use of N-stearoyl-D-serine-methyl ester (yield: 12%, [α]²⁵D=+8.8 (c 2.5, MeOH)). When analyzing the structure of the synthesized compound, the ¹H-NMR and ¹³C-NMR results were identical to those of N-stearoyl-O-phosphocholine-L-serine methyl ester.

### EXAMPLE 4

### Synthesis of N-stearoyl-O-phosphocholine-D-serine methylhydroxy (CHJ-0014)

(i) Synthesis of D-serine methyl ester hydrochloride

D-serine methyl ester hydrochloride was synthesized according to the same method as in Example 1 for synthesis of L-serine methyl ester hydrochloride, except for use of D-serine instead of L-serine (yield: 99%, melting point: 163-164° C., [α]²⁵D=4.3 (c 1.8, EtOH)). When analyzing the structure of the synthesized compound, the FTIR, ¹H-NMR and ¹³C-NMR results were identical to those of L-serine methyl ester hydrochloride.

(ii) Synthesis of N-stearoyl-D-serine methyl ester

N-stearoyl-D-serine methyl ester was synthesized according to the same method as in Example 1 (ii) for synthesis of N-stearoyl-L-serine methyl ester, except for use of D-serine-methyl ester hydrochloride (yield: 88%, melting point: 82-83° C., [α]²⁵D=−15.7 (c 2.0, CHCl₃)). When analyzing the structure of the synthesized compound, the FTIR, ¹H-NMR and ¹³C-NMR results were identical to those of N-stearoyl-L-serine methyl ester.

(iii) Synthesis of N-stearoyl-O-phosphocholine-D-serine methyl ester (CHJ-0012)

N-stearoyl-O-phosphocholine-D-serine methyl ester was synthesized according to the same method as in Example 1 (iii) for synthesis of N-stearoyl-O-phosphocholine-L-serine methyl ester, except for use of N-stearoyl-D-serine-methyl ester (yield: 12%, [α]²⁵D=+8.8 (c 2.5, MeOH)). When analyzing the structure of the synthesized compound, the ¹H-NMR and ¹³C-NMR results were identical to those of N-stearoyl-O-phosphocholine-L-serine methyl ester.

(iv) Synthesis of N-stearoyl-O-phosphocholine-D-serine methylhydroxy (CHJ-0014)

N-stearoyl-O-phosphocholine-D-serine methylhydroxy was synthesized according to the same method as in Example 2 (iv) for synthesis of N-stearoyl-O-phosphocholine-L-serine methylhydroxy (CHJ-0013), except for use of N-stearoyl-O-phosphocholine-D-serine-methyl ester (yield: 53%, [α]²⁵D=−5.3 (c 2.0, CH₂Cl₂/MeOH)). When analyzing the structure of the synthesized compound, the FTIR, ¹H-NMR and ¹³C-NMR results were identical to those of CHJ-0013.

### EXPERIMENTAL EXAMPLES

Cell Culture

Primary osteoblasts, bone marrow cells and osteoclast precursor cells were cultured in α-Minimum Essential Medium (α-MEM, Gibco BRL) supplemented with 10% (v/v) fetal bovine serum (Gibco BRL), and 1× antibiotics containing penicillin/streptomycin (Gibco BRL).

### Experimental Example 1

### Evaluation of Inhibitory Activity of CHJ-0014 on Differentiation of Osteoclasts: Co-Culture of Osteoblasts and Bone Marrow Cells

Until now, osteoclast cell lines have not been established, making it difficult to study differentiation of osteoclasts. Osteoclasts originate from hematopoietic stem cells derived from bone marrow, and differentiate with the help of osteoblasts/stromal cells. For these reasons, the co-culture system of osteoblasts and bone marrow cells is used for differentiation of osteoclasts. In these Experimental Examples, a differentiation system of bone marrow cells to osteoclasts was established by co-culture of osteoblasts and bone marrow cells, and, in this system, the CHJ-0014 compound was evaluated for its inhibitory effect on differentiation of osteoclasts.

1) Isolation of Osteoblasts

After sacrificing one-day postnatal ICR mice and disinfecting them in 70% ethanol, calvarias were collected using surgical scissors and forceps, cut into several pieces, and transferred into 3× HBSS contained in a 60-mm culture dish. 0.1% collagenase (Gibco BRL) and 0.2% dispase (Boehringer Mannheim) were added to the solution, followed by incubation at 37° C. for 15 min. The treatment of calvarias with collagenase and dispase was repeated 4 more times. From the second treatment of calvarias with collagenase and dispase, the solution was centrifuged after each treatment at 1600 rpm for 5 min to harvest cells, thereby collecting osteoblasts. The collected osteoblasts were plated onto a 100-mm culture dish at a density of about 1-2×10⁶ cells, and cultured in 15 ml of α-MEM supplemented with 10% FBS for 3 days. Thereafter, the proliferated osteoblasts were aliquotted into cryo-vials, and stored in liquid nitrogen until use in co-culture of osteoblasts and bone marrow cells.

2) Isolation of bone marrow cells

After sacrificing 6-7 week ICR female mice by cervical dislocation and disinfecting their rear legs in 70% ethanol, tibias were isolated aseptically. The isolated tibias were placed into 3× HBSS (Gibco BRL), and soft tissue was completely removed. Both ends of tibias were cut, and 1×α-MEM was injected into bone marrow and then sucked up using a 1 ml syringe, collecting bone marrow cells. After sufficient suspending by pipetting, the collected bone marrow cells were centrifuged at 1600 rpm for 5 min to harvest cells. The resulting pellet (bone marrow cells and erythrocytes) was suspended in about 15-20 ml of ACK buffer (155 mM NH₄Cl, 11 mM KHCO₃, 0.01 mM EDTA). After incubation for 2 min, phosphate buffer was added to the cell suspension to minimize damage to bone marrow cells and lyse erythrocytes.

Thereafter, the cell suspension was centrifuged (1600 rpm, 5 min), and the resulting cell pellet was suspended in 10% FBS-containing α-MEM.

3) Co-culture of bone marrow cells and osteoblasts

The isolated and cultured osteoblasts and bone marrow cells were co-cultured, as follows. Bone marrow cells and osteoblasts were plated onto a 48-well plate at a density of 2×10⁵ cells and 2×10⁴ cells per well, respectively, and co-cultured in 10% FBS-containing x-MEM. After adding vitamin D3 (10⁻⁸ M) and PGE 2 (Prostaglandin E 2, 10⁻⁶ M) to the culture medium, the cells were treated with the CHJ-0014 compound at various concentrations of 1.65 μM, 3.3 μM and 6.6 μM. A co-culture group not treated with the CHJ-0014 compound was used as a control. When the culture medium was exchanged with a fresh medium after incubation for 3 days, vitamin D3 (10⁻⁸ M) and PGE 2 (10⁻⁶ M) were again added to the medium, and the CHJ-0014 compound was also added to the medium at the same concentrations as above.

After incubation for 6 days, the culture medium was removed, and completely differentiated osteoclasts were fixed with 10% formalin for 5 min. After removing formalin, the fixed osteoclasts were treated with 0.1% Triton X-100 for 10 sec. After removing Triton X-100, the cells were stained with TRAP (tartrate-resistant acid phosphatase) for 5 min. TRAP staining was carried out using the Leukocyte Acid Phosphatase Kit (Sigma, Cat. No. 387-A). After eliminating the TRAP staining solution, the cells were washed with distilled water twice, dried, and TRAP-positive osteoclasts were counted under an optical microscope (×100).

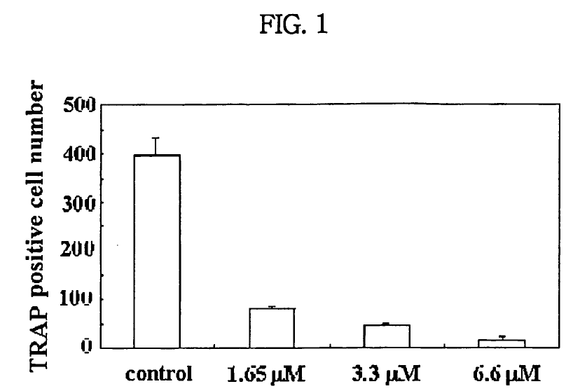

As a result, in the control, TRAP-positive cell number was 397+36.75. In the groups treated with the CHJ-0014 compound of 1.65 μM, 3.3 μM and 6.6 μM, TRAP-positive osteoclast cell numbers were 80±6.1, 46±4.7 and 16±6.4, respectively (FIG. 1).

These results indicate that the CHJ-0014 compound inhibits differentiation of osteoclasts in co-culture of bone marrow cells and osteoblasts in a dose-dependent manner.

### Experimental Example 2

### Evaluation of Inhibitory Activity of CHJ-0014 on Differentiation of Osteoclasts: Culture of Bone Marrow Cells

The inhibitory activity of the CHJ-0014 compound on osteoclast differentiation, identified in the Experimental Example 1, possibly resulted from the fact that the CHJ-0014 compound affects osteoblasts to indirectly inhibit osteoclast differentiation. In this test, to evaluate this possible scenario, only bone marrow cells were treated with a recombinant ODF protein and inhibitory activity of the CHJ-0014 compound on osteoclast differentiation was investigated in culture of only bone marrow cells. Bone marrow cells, isolated according to the same method as in Experimental Example 1, were plated onto a 48-well plate at a density of 4×10⁵ cells, and cultured in 10% FBS-containing α-MEM. After adding ODF (50 ng/ml) and M-CSF (30 ng/ml) to the medium, the cells were treated with the CHJ-0014 compound at various concentrations of 0.825 μM, 1.65 μM, 3.3 μM and 6.6 μM. When the culture medium was exchanged with a fresh medium after incubation for 3 days, the cells were again treated with the identical amount of ODF, M-CSF and CHJ-0014. After incubation for 6 days, the culture medium was exchanged with a fresh medium, and the cells were further cultured for 2 more days.

Completely differentiated osteoclasts were identified by TRAP staining. TRAP staining was carried out using the Leukocyte Acid Phosphatase Kit (Sigma, Cat. No. 387-A). The culture medium was removed, and completely differentiated osteoclasts were fixed with 10% formalin for 5 min. After removing formalin, the fixed osteoclasts were treated with 0.1% Trypton X-100 for 10 sec. After removing Trypton X-100, the cells were stained with a TRAP staining solution in the kit for 5 min. After eliminating the TRAP staining solution, the cells were washed with distilled water twice, dried, and TRAP-positive osteoclasts were counted under an optical microscope (×100).

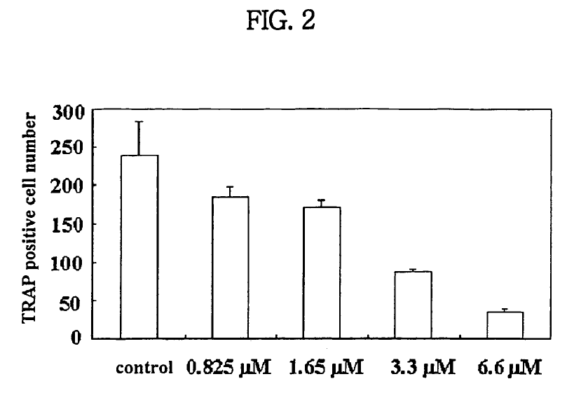

As a result, in case of a control not treated with CHJ-0014, TRAP-positive cell number was 240±44. In the groups treated with the CHJ-0014 compound of 0.825 μM, 1.65 μM, 3.3 μM and 6.6 μM, TRAP-positive osteoclast cell numbers were 185±12.9, 172+9.3, 87+3.5 and 36+3.6, respectively (FIG. 2). These results indicate that the CHJ-0014 compound inhibits osteoclast differentiation induced by ODF in bone marrow cells in a dose-dependent manner.

### Experimental Example 3

### Evaluation of Inhibitory Activity of CHJ-0014 on Differentiation of Osteoclast Precursor Cells

Osteoclast progenitors derived from bone marrow cells migrate eventually to bone, differentiate to osteoclast precursors therein, and further differentiate to active osteoclasts having bone resorption ability and participate in bone resorption. That is, osteoclast precursor cells present in bone are at a different differentiation stage from the active osteoclasts. Recently, a cytokine playing a critical role in osteoclast differentiation was identified to be ODF (also called OPGL or RANKL), and a recombinant ODF protein can be produced on a large scale, thereby facilitating osteoclast differentiation by primary cell culture.

Osteoclast precursor cells were isolated from iliac bone. After sacrificing 5-6 week ICR female mice by cervical dislocation and disinfecting their rear legs in 70% ethanol, tibias and femurs were isolated aseptically. After removing soft tissue according to the same method as in Example 2 from the isolated tibias and femurs, 3× HBSS (Gibco BRL) was injected into bone marrow several times, thereby collecting bone marrow cells, as follows. Bone was cut into small pieces using a surgical scissor, and incubated in a collagenase solution (1 mg/ml collagenase type II, 0.05% trypsin, 4 mM EDTA, Gibco BRL) contained in a culture dish at 37° C. for 15 min. The collagenase treatment was carried out 4 more times. After being treated three times, bone pieces were further cut into a smaller size. After one more treatment with collagenase, the bone pieces were washed with 3× HBSS five times. After completely removing the enzyme solution by centrifugation, the bone pieces were suspended in pre-cooled α-MEM, and placed onto ice for 15 min. After vortexing for 1 min, an equal volume of α-MEM was added to the bone suspension, followed by incubation on ice for 15 min. The bone suspension was filtered through a sterilized mass, and the obtained cells were cultured in 10% FBS-containing α-MEM.

The isolated and cultured osteoclast precusor cells were plated onto a 48-well plate at a density of 0.5×10⁶ cells. After adding ODF (100 ng/ml) and M-CSF (30 ng/ml) to the medium, the cells were treated with the CHJ-0014 compound at various concentrations of 0.825 μM, 1.65 μM, 3.3 μM and 6.6 μM. A group not treated with the CHJ-0014 compound was used as a control. When the culture medium was exchanged with a fresh medium after incubation for 3 days, the cells were again treated with the identical amount of ODF, M-CSF and CHJ-0014. On day 6 after culturing, the culture medium was exchanged with a fresh medium, according to the same method as in on day 3. On day 9, TRAP staining was carried out, and TRAP-positive osteoclasts were counted under an optical microscope (×100).

As a result, in case of the control, TRAP-positive cell number was 344±43.7.

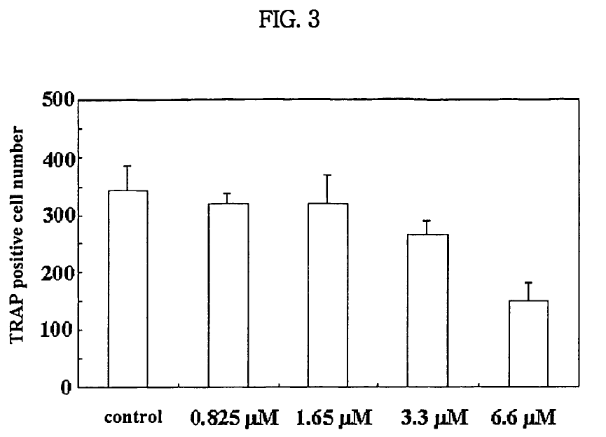

In the groups treated with the CHJ-0014 compound of 0.825 μM, 1.65 μM, 3.3 μM and 6.6 μM, TRAP-positive osteoclast cell numbers were found to be 318±19.3, 318±50.4, 267±24 and 152±29.1, respectively (FIG. 3). These results indicate that the CHJ-0014 compound inhibits osteoclast differentiation during differentiation of osteoclast precursors isolated from bone in a dose-dependent manner.

### Experimental Example 4

### Evaluation of Cytotoxic Activity of CHJ-0014

The CHJ-0014 compound was evaluated in various cell types for cytotoxicity by MTT (3-[4,5-dimethylthiazole-2-yl]-2,5-diphenyl tetrazolium bromide) assay

Osteoblasts prepared in Experimental Experiment 3, murine bone marrow cells, peritoneal macrophages, and the human embryonic kidney cell line 293T were used in MTT assay. The various cell types were plated onto a 96-well plate at a density of 1.5×10⁴, 1×10⁵, 1×10⁵ and 1.5×10⁴ cells per well, respectively, and treated with the CHJ-0014 compound at a concentration of 6.6 μM, which was the highest concentration in the osteoclast differentiation tests. After incubation for 24 hrs, 50 μl of a mixture for MTT analysis (mixture of 1 ml MTT labeling reagent and 0.2 ml electron coupling reagent) was added to each well, followed by incubation at room temperature for 4 hrs.

Absorbance was measured at 450 nm and 630 nm using an ELISA reader (Bio-Tek Instrument, Winooski, Vt.). This MTT assay was repeated two more times.

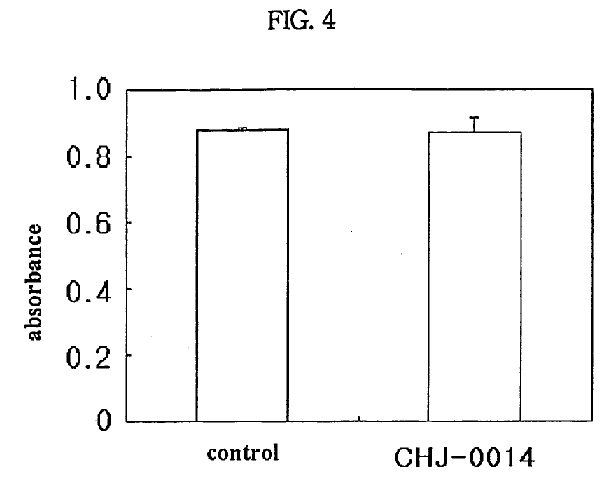

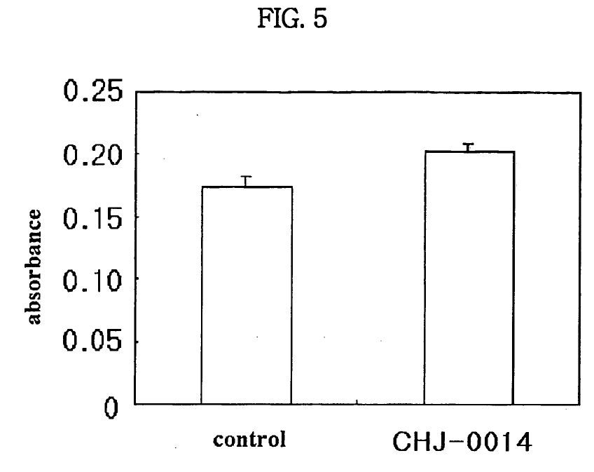

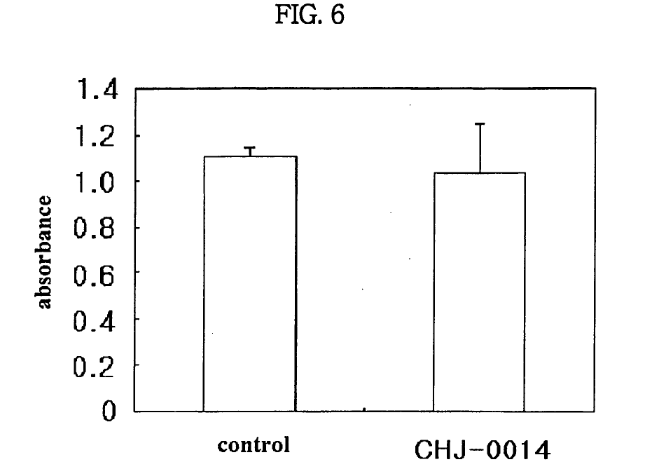

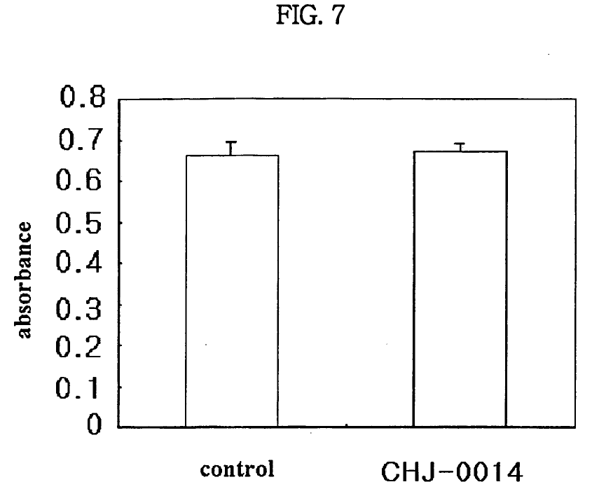

As a result, the compound CHJ-0014 compound did not show cytotoxicity in any of osteoblasts, bone marrow cells, peritoneal macrophages and 293T cells (FIGS. 4 to 7).

### Experimental Example 5

### Evaluation of Effect of CHJ-0014 on NF-κB Activity

The CHJ-0014 compound was investigated for its inhibitory effect on the activity of the transcription factor NF-κB participating in osteoclast differentiation by performing EMSA (electrophoresis mobility shift assay). Osteoclasts were prepared according to the same method as in Experimental Experiment 3, and incubated in hypotonic lysis buffer (10 mM HEPES, pH 7.9, 1.5 mM MgCl₂, 10 mM KCl, 0.5 mM DTT, 0.5 mM PMSF) on ice for 10 min. The osteoclast-containing solution was transferred into a microcentrifuge, supplemented with 0.1% NP40, and incubated for 15 min with occasional agitation. After centrifugation (4,000 rpm, 15 min), the resulting cell pellet was suspended in 15 μl of high salt buffer (20 mM HEPES, pH 7.9, 420 mM NaCl, 25% glycerol, 1.5 mM MgCl₂, 0.2 mM EDTA, 0.5 mM PMSF, 0.5 mM DTT), and placed onto ice for 20 min. 75 μl of storage buffer (20 mM HEPES, pH 7.9, 100 mM NaCl, 20% glycerol, 0.2 mM EDTA, 0.5 mM PMSF, 0.5 mM DTT) was added to the cell suspension, and incubated with agitation for 10 sec. After centrifugation (14,000 rpm, 20 min), the resulting supernatant was subjected to protein quantitative assay. Protein assay was performed using a DC Protein Assay Kit (Bio-Rad).

A NF-κB-specific oligomer (5′-AGTTGAGGGGACTTTCCCA GGC-3′, Santa Cruz) was radiolabeled using [γ-³²P]ATP and Klenow fragment, and used as a probe.

10 μg of protein and about 20,000 cpm of the ³²P-labeled probe were added to 20 μl of 1 μg of poly(dIdC)-containing reaction buffer (10 mM Tris-HCl, 50 mM KCl, 1 mM EDTA, 5% glycerol, 2 mM DTT), followed by incubation at room temperature for 30 min.

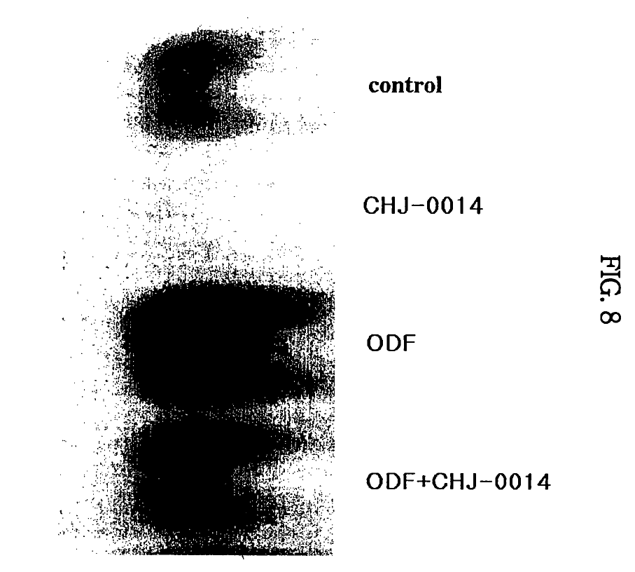

As a result, the CHJ-0014 compound was found to suppress activation of the transcription factor NF-κB by osteoclast differentiation factor (ODF) (FIG. 8).

### Experimental Example 6

### Evaluation of Inhibitory Activity of CHJ-0013 and CHJ-0014 on Osteoclast Differentiation: Co-Culture of Bone Marrow Cells and Osteoblasts

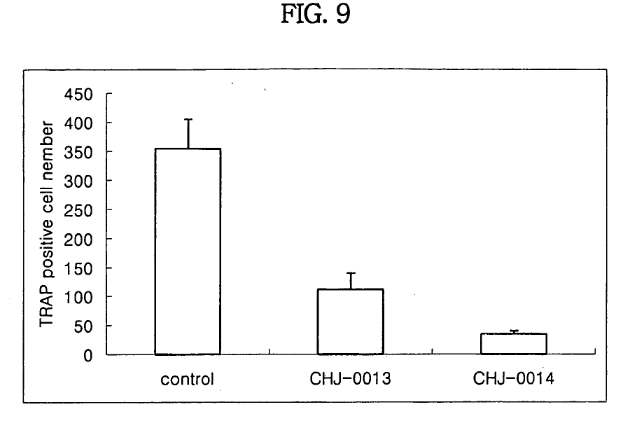

The CHJ-0013 and CHJ-0014 compounds were evaluated for inhibitory activity on osteoclast differentiation in co-culture of bone marrow cells and osteoblasts according to the same procedure as in the Experimental Experiment 1, except for use of the CHJ-0013 and CHJ-0014 compounds at an amount of 4 μM. The results are given in FIG. 9. The CHJ-0013 and CHJ-0014 compounds were found to inhibit osteoclast differentiation in co-culture of bone marrow cells and osteoblasts in a dose-dependent manner.

### FORMULATION EXAMPLE

The components, below, were mixed and compressed to formulate into tablets according to the conventional method common in the pharmaceutical formulation field.

| | | |
|---|---|---|
| CHJ-0014 | 25 | mg |
| Lactose | 66 | mg |
| Microcrystalline glucose NF | 20 | mg |
| Sodiumstarch glucholate NF | 2.99 | mg |
| Talc USP | 1.5 | mg |
| Stearate magnesium | 0.7 | mg |

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### INDUSTRIAL APPLICABILITY

As described hereinbefore, the N-acylated lysophosphatidylcholine compound represented by Formula 1 has high inhibitory activity on osteoclast differentiation, as well as no cytotoxicity. Therefore, a pharmaceutical composition comprising the N-acylated lysophosphatidylcholine compound represented by Formula 1 is believed to be very useful for prevention or treatment of metabolic bone diseases.

1. A pharmaceutical composition for treating or preventing metabolic bone diseases, comprising a pharmaceutically effective amount of an N-acyl-O-phosphochloine-L-serine methylester compound represented by Formula 1a or its pharmaceutically acceptable salt, alone or in combination with a pharmaceutically acceptable carrier: 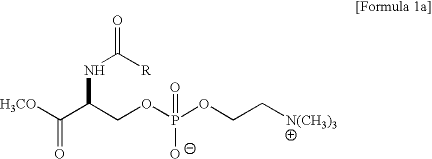
wherein R is a saturated or unsaturated fatty acid having 14 to 20 carbon atoms.

2. A pharmaceutical composition for treating or preventing metabolic bone diseases, comprising a pharmaceutically effective amount of N-acyl-O-phosphocholine-D-serine methylester compound represented by Formula 1b or its pharmaceutically acceptable salt, alone or in combination with a pharmaceutically acceptable carrier: 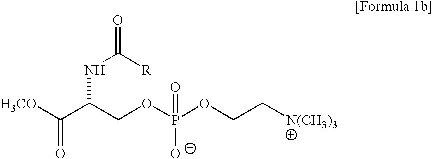
wherein, R is a saturated or unsaturated fatty acid having 14 to 20 carbon atoms.

3. A pharmaceutical composition for treating or preventing metabolic bone diseases, comprising a pharmaceutically effective amount of N-acyl-O-phosphocholoine-D-serine methylhydroxy compound represented by Formula 1c or its pharmaceutically acceptable salt, alone or in combination with a pharmaceutically acceptable carrier: 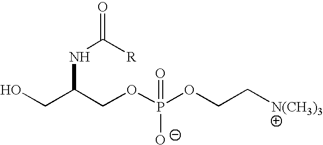
wherein, R is a saturated or unsaturated fatty acid having 14 to 20 carbon atoms.

4. A pharmaceutical composition for treating metabolic bone diseases, comprising a pharmaceutically effective amount of a N-acyl-O-phosphocholine-D-serine methylhydroxy compound represented by Formula 1d or its pharmaceutically acceptable salt, alone or in combination with a pharmaceutically acceptable carrier: 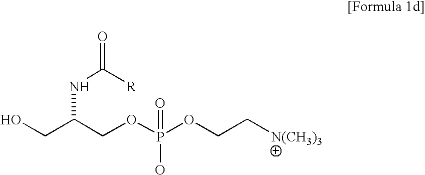
wherein, R is a saturated or unsaturated fatty acid having 14 to 20 carbon atoms.

5. The pharmaceutical composition as set forth in claim 3 wherein the R is a stearoyl group having 17 carbon atoms.

6. The pharmaceutical composition as set forth in claim 1 wherein the metabolic bone disease is selected from the group consisting of osteoporosis, metastatic bone lesions, primary bone tumors, rheumatoid or degenerative arthritis, periodontal disease, inflammatory periodontal disease with alveolar bone destruction, inflammatory bone resorption disease, and Pazet's disease.

7. A method of preparing a compound represented by Formula 1 comprising the steps of:
(a) reacting serine with methanol and hydrochloric acid to produce serine methylester hydrochloride;
(b) reacting serine methylester hydrochloride with N-methylmorpholine, a saturated or unsaturated fatty acid having 14 to 20 carbon atoms, 1-hydroxybenzotriazole and 1,3-dicyclohexylcarbodiimide to produce N-acyl-serine methylester;
(c) reacting the N-acyl-serine methylester with N-diisopropylethyl amine and ethylene chlorosphosphite,
(d) reacting the formed compoumd at the step (c) with trimethylamine to produce N-acyl-O-phosphocholine-serine methylester; and
(e) reacting the N-acyl-O-phosphochloine-serine methylhydroxy, 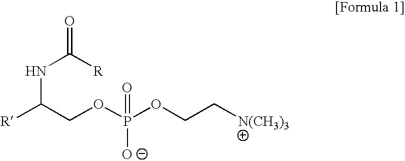
wherein, R is saturated or unsaturated fatty acid having 14 to 20 carbon atoms, and R′ is methoxycarbonyl or hydroxymethyl group.

8. The method as set forth in claim 7, wherein the compound of Formula 1 is in an L-stereoisomeric form, and the method comprises the steps of:
(a) reacting L-serine with methanol and hydrochloric acid to produce L-serine methylester hydrochloride;
(b) reacting the L-serine methylester hydrochloride with N-methylmorpho line, a saturated or unsaturated fatty acid and having 14 to 20 carbon atoms, 1-hydroxybenzotriazole and 1,3-dicyclohexylcarbodiimide to produce N-acyl-L-methylester,
(c) reacting the N-acyl-serine methylester with N-diisopropylethyl amine and ethylene chlorophosphite, and trimethylamine to produce N-acyl-O-phosphocholine-L-serine methylester; and
(d) reacting the N-acyl-O-phosphochlonine-L-serine methylester with lithiumaluminumhydride to produce N-acyl-O-phosphochloine-L-serine methylhydroxy: 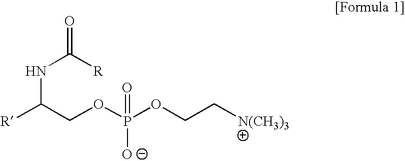
wherein R is a saturated or unsaturated fatty acid having 14 to 20 carbon atoms, and R′ is methoxycarbonyl or hydroxylmethyl group.

9. The method as set forth in claim 7, wherein the compound of Formula 1, below, is in a D-stereoisomeric form, and the method comprises the steps of:
(a) reacting D-serine with methanol and hydrochloric acid to produce D-serine methylester hydrochloride;
(b) reacting the D-serine methylester hydrochloride with N-methylmorpholine, a saturated or unsaturated fatty acid having 14 to 20 carbon atoms, 1-hydroxybenzotriazole and 1,3-dicyclohexylcarbodiimide to produce N-acyl-D-serine methylester;
(c) reacting the N-acyl-D-serine methylester with N-diisopropylethyl amine and ethylene chlorophosphite, and then trimethylamine to produce N-acyl-O-phosphochloine-D-serine methylester; and
(d) reacting the N-acyl-O-phosphochloine-D-serine methylester with lithiumaluminumhydride to produce N-acyl-O-phospohocholine-D-serine methylhydroxy, 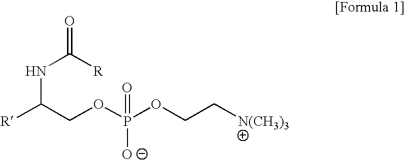
wherein, R is saturated or unsaturated acid having 14 to 20 carbon atoms, and R′ is methoxycarbonyl or hydroxymethyl group.

10. The pharmaceutical composition of claim 4 wherein the R is a stearoyl group having 17 carbon atoms.

11. A pharmaceutical composition of claim 2
wherein the metabolic bone disease is selected from the group consisting of osteoporosis, metastatic bone lesions, primary bone tumors, rheumatoid or degenerative arthritis, periodontal disease, inflammatory periodontal disease with alveolar bone destruction, inflammatory bone resorption disease, and Pazet's disease.

12. A pharmaceutical composition of claim 3
wherein the metabolic bone disease is selected from the group consisting of osteoporosis, metastatic bone lesions, primary bone tumors, rheumatoid or degenerative arthritis, periodontal disease, inflammatory or periodontal disease with alveolar bone destruction, inflammatory bone resorption, and Pazet's disease.

13. A pharmaceutical composition of claim 4
wherein the metabolic bone disease is selected from the group consisting of osteoporosis, metastatic bone lesions, primary bone tumors, rheumatoid or degenerative arthritis, periodontal disease, inflammatory periodontal disease with alveolar bone destruction, inflammatory bone resorption disease, and Pazet's disease.
